(19) United States
(12) Patent Application Publication
(10) Pub. No.: US 2026/0191522 A1
(43) Pub. Date: Jul. 9, 2026

(54) BONE ANCHOR, IMPLANTATION HANDLE, AND IMPLANTATION SYSTEM

(71) Applicant: THE SECOND AFFILIATED HOSPITAL ZHEJIANG UNIVERSITY SCHOOL OF MEDICINE, Hangzhou (CN)

(72) Inventor: Weiliang SHEN, Hangzhou (CN)

(21) Appl. No.: 19/132,813

(22) PCT Filed: Jan. 30, 2024
(86) PCT No.: PCT/CN2024/074624

§ 371 Date: May 25, 2025

(30) Foreign Application Priority Data
Jun. 20, 2023 (CN) 202321572920.1
Jun. 20, 2023 (CN) 202321572932.4

(51) Int. Cl. A61B 17/04; A61B 17/00

(52) U.S. Cl. CPC A61B 17/0401; A61B 2017/0042; A61B 2017/0409; A61B 2017/0414; A61B 2017/044

(57) ABSTRACT

A bone anchor, an implantation handle, and an implantation system are provided. The anchor includes an anchor body with axially extending through-lumen and, from its proximal toward its distal end, including an upper, middle, and lower section. The outer diameter of the middle section is smaller than that of the upper section. The upper section tapers conically and has a first helical thread. A recess in the distal face of the upper section is in communication with the lumen. The middle section has a cylindrical external thread, and the lower section has an open-work thread formed by hollow thread flanges. The cylindrical thread and the open-work thread together define a second helical thread. Multiple reinforcing ribs are disposed on the inner side of the open-work thread around the lumen. This construction reduces the risk of anchor pull-out in osteoporotic bone.

### CROSS-REFERENCE TO RELATED APPLICATION

This application is a national stage application of International Patent Application No. PCT/CN2024/074624, filed Jan. 30, 2024, which claims two priorities of the Chinese Patent Application No. 202321572920.1 and the Chinese Patent Application No. 202321572932.4, filed with the China National Intellectual Property Administration on Jun. 20, 2023, and entitled “BONE ANCHOR, IMPLANTATION HANDLE, AND IMPLANTATION SYSTEM”, all of which are incorporated by references in their entities.

### TECHNICAL FIELD

The present disclosure relates to the technical field of medical devices, and in particular to a bone anchor, an implantation handle, and an implantation system.

### BACKGROUND

When rotator cuff or other muscle tendons are injured, surgery is often needed to repair the injury and promote rehabilitation. At present, in clinical practice, for completely separated cases or certain partially separated cases, bone anchors are generally used to reconnect soft tissue to bone. The anchors are embedded into the bone substance to fix the lacerated tendinous tissue, thereby achieving tendon-bone healing.

For osteoporotic patients, due to their low bone density, during the implantation of bone anchors, the loose bone substance increases the risk of bone anchor pulled out when the bone anchor is tensioned by the suture. For this reason, large-sized bone anchors are used in many practices, but there is still the risk for the large-sized bone anchors to be pulled out. The pullout resistance of the bone anchor can be enhanced by injecting bone cement, but the injection of the bone cement and the implantation of the bone anchor often require different operating systems. In this process, there are problems that the bone cement penetrates into the bone anchor and the bone substance around the bone anchor is poor or excessive. Meanwhile, there is the risk that the bone anchor is taken out by the bone cement ejector before the bone cement hardens, which affects the fixation effect of the anchor.

### SUMMARY

An objective of the present disclosure is to provide a bone anchor, an implantation handle, and an implantation system, thus solving the technical problems in the prior art.

To achieve the objective above, the present disclosure provides the following technical solutions:

The present disclosure provides a bone anchor, and the bone anchor includes an anchor body. An interior of the anchor body is provided with a through hollow cavity in an axial direction thereof. In a direction from a tail to a head of the anchor body, the anchor body is divided into an upper section, a middle section and a lower section. An outer diameter of the middle section is smaller than an outer diameter of the upper section.

The outer diameter of the upper section gradually decreases in the direction from the tail to the head of the anchor body, and the upper section is conical as a whole. A surface of the upper section is provided with first screw threads. An end face, away from the middle section, of the upper section is provided with a groove, and the groove is in communication with the hollow cavity.

The middle section is provided with cylindrical screw threads, the lower section is provided with hollowed-out thread teeth, and the cylindrical screw threads and the hollowed-out thread teeth form second screw threads.

Inner sides of the hollowed-out thread teeth are provided with multiple reinforcing ribs around an axis of the anchor body.

In an embodiment, the cylindrical screw threads and the hollowed-out thread teeth form a continuous threaded structure.

In an embodiment, a joint of the upper section and the middle section presents a conical frustum-shaped transition, and four independent suture holes are arranged between an outer surface of the conical frustum-shaped transition and an end surface of the upper section in the axial direction of the anchor body.

In an embodiment, each of the upper section, the middle section and the lower section accounts for one third of a total length of the anchor body, and the multiple reinforcing ribs are connected with the middle section.

In an embodiment, the multiple reinforcing ribs are uniformly arranged.

In an embodiment, multiple holes in communication with the hollow cavity are arranged between adjacent grooves of the cylindrical screw threads of the middle section.

In an embodiment, the multiple reinforcing ribs include two or four reinforcing ribs.

In an embodiment, a radial section of the groove is quincunx or hexagonal.

In an embodiment, a taper of the upper section is smaller than a taper of the conical frustum-shaped transition.

The present disclosure further provides an implantation handle, and the implantation handle includes a guide sleeve component, a handle component and a drive rod.

The guide sleeve component is provided with a guide sleeve and a gripping part fixedly connected with the guide sleeve, and the gripping part is provided with a connecting slot. One end, away from the gripping part, of the guide sleeve is provided with a non-return part.

A handle body in the handle component is fixedly connected with the drive rod, the handle body is provided with an engagement part, and the drive rod is inserted into the guide sleeve.

The engagement part is detachably connected with the connecting slot.

In an embodiment, a curved sidewall of the connecting slot is provided with a first vertical guide groove and a first horizontal sliding chute which are in communication with each other, and a second vertical guide groove and a second horizontal sliding chute which are in communication with each other.

In an embodiment, the engagement part of the handle body is provided with a first locking post and a second locking post. The first locking post slides into or out of the first vertical guide groove and the first horizontal sliding chute. The second locking post slides into or out of the second vertical guide groove and the second horizontal sliding chute.

In an embodiment, one end, away from the engagement part, of the drive rod is provided with a driving part.

In an embodiment, a center of the drive rod is provided with a through guide hole in an axial direction thereof.

In an embodiment, an outer diameter of the non-return part gradually decreases in a direction away from the gripping part, and the non-return part is conical as a whole.

In an embodiment, one end, away from the engagement part, of the handle body is provided with an external threaded connecting part.

In an embodiment, the implantation handle further includes a core rod, the core rod is configured to be insertable into the drive rod. The core rod is provided with an operation cap, and the operation cap is provided with an internal threaded connecting part.

In an embodiment, one end, away from the operation cap, of the core rod is provided with an inserting tip.

The present disclosure further provides an implantation system, including using the implantation handle.

Compared with the prior art, the present disclosure has the following technical effects:
(1) The overall design of the bone anchor enables the bone anchor to have good pulling resistance in osteoporotic bone, which is more suitable for osteoporotic patients. Suture holes are formed in a conical frustum between an upper section and a middle section, and thus the friction between the suture and the bone can be reduced to lower the risk of suture fracture. The design of the conical frustum shape and the tapered screw thread of the upper section of the anchor can make the upper section of the anchor smoothly insert into cortical bone, thus reducing the damage to the cortical bone of the osteoporotic patient.
(2) The lower section of the bone anchor is provided with hollowed-out thread teeth. It should be pointed out that on the one hand, the hollowed-out thread teeth can ensure that cancellous bone can grow inside the hollowed-out thread teeth to facilitate the growth of the bone in the healing process, which can further enhance the pullout strength of the anchor and reduce the possibility of the falling of the anchor; on the other hand, after bone cement is injected into a hollow cavity of the anchor, it is beneficial for the bone cement to flow out of the hollowed-out structure to fill a gap between the anchor and the bone, thus enhancing the pullout strength of anchor in the cancellous bone.
(3) After the implantation of the bone anchor and the injection of the bone cement are completed by using the implantation handle, a gripping part in a guide sleeve component is rotated to make a connecting slot of the gripping part separated from an engagement part of a handle body. In the process of taking out the handle component, a non-return part of the guide sleeve is always in contact with a groove on the upper section of the bone anchor, thus preventing a drive rod from taking out the bone anchor from the bone when withdrawn with the handle component.

### BRIEF DESCRIPTION OF THE DRAWINGS

To describe the technical solutions of the embodiments of the present disclosure or in the prior art more clearly, the following briefly introduces the accompanying drawings required and used in the embodiments. Apparently, the accompanying drawings in the following description show merely some embodiments of the present disclosure, and those skilled in the art can still derive other drawings from these accompanying drawings without creative efforts.

FIG. 1 is a front view of a bone anchor according to an embodiment of the present disclosure;

FIG. 2 is a top view of a bone anchor according to an embodiment of the present disclosure;

FIG. 3 is a sectional view of a bone anchor according to an embodiment of the present disclosure;

FIG. 4 is a structural diagram of an implantation handle according to an embodiment of the present disclosure;

FIG. 5 is a structural diagram of a bone anchor according to an embodiment of the present disclosure;

FIG. 6 is a structural diagram of a guide sleeve component according to an embodiment of the present disclosure;

FIG. 7 is a diagram of a partially enlarged structure at part A in FIG. 6;

FIG. 8 is a structural diagram of a handle component according to an embodiment of the present disclosure;

FIG. 9 is a top view of a handle component according to an embodiment of the present disclosure;

FIG. 10 is a structural diagram of a drive rod according to an embodiment of the present disclosure;

FIG. 11 is a diagram of a partially enlarged structure at part B in FIG. 10;

FIG. 12 is a top view of a drive rod according to an embodiment of the present disclosure;

FIG. 13 is a structural diagram of a core rod according to an embodiment of the present disclosure;

FIG. 14 is a diagram of a partially enlarged structure at part C in FIG. 13;

FIG. 15 is a first structural diagram of an operation cap according to an embodiment of the present disclosure;

FIG. 16 is a second structural diagram of an operation cap according to an embodiment of the present disclosure.

### REFERENCE NUMERALS IN THE DRAWINGS

1—anchor body; 11—hollow cavity; 12—suture hole; 2—upper section upper section; 3—middle section; 4—lower section; 21—groove; 31—hole; 41—hollowed-out thread teeth; 42—reinforcing rib;
10—bone anchor; 101—anchor body; 102—head of anchor; 103—tail of anchor; 104—groove;
20—guide sleeve component; 201—guide sleeve; 202—non-return part; 203—connecting part; 204—gripping part; 2041—connecting slot; 2042—guide hole; 2043—first vertical guide groove; 2044—second vertical guide groove; 2045—first horizontal sliding chute; 2046—second horizontal sliding chute;
30—handle component; 301—handle body; 302—external threaded connecting part; 303—engagement part; 304—first locking post; 305—second locking post; 306—injection hole;
40—drive rod; 401—connecting part; 402—driving part; 403—guide hole;
50—core rod; 501—connecting end part; 502—inserting tip; 503—operation cap; 5031—Z-shaped operation part; 5032—internal threaded connecting part.

### DETAILED DESCRIPTION OF THE EMBODIMENTS

In order to make the technical problem to be solved in the present disclosure, technical solutions and beneficial effects of the present disclosure more clearly, the present disclosure is further described in detail below with reference to the accompanying drawings and embodiments. It should be understood that specific embodiments described herein are only used to illustrate rather than limiting the present disclosure.

It should be noted that when a component is said to be “fixed” or “disposed” on another component, it can be directly or indirectly located on another component. When an component is said to be “connected” to another component, it can be directly or indirectly connected to another component. The orientations or locations indicated by the terms “upper”, “lower”, “left”, “right”, “front”, “back”, “vertical”, “horizontal”, “top”, “bottom”, “inside”, “outside” and the like are the orientations or locations shown in the drawings, which are only used for the convenience of description, and cannot be construed as the limitation to the technical solution. The terms “first” and “second” are only used for the convenience of description and are not to be construed as indicating or implying relative importance or implying the number of the indicated technical features. “A plurality of” means two or more, unless otherwise specifically defined.

### Embodiment I

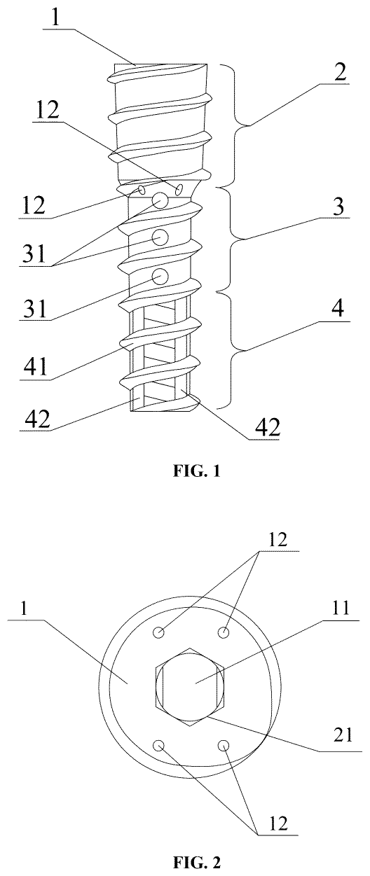

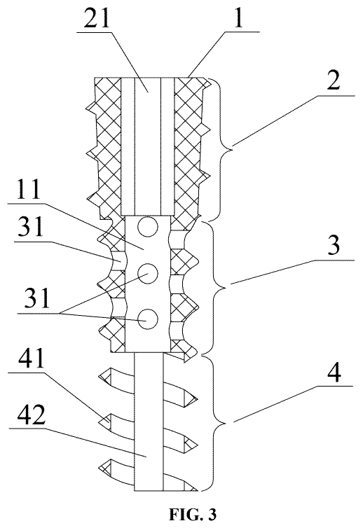

Please referring to FIG. 1 to FIG. 3, a bone anchor is provided in this embodiment, and the bone anchor includes an anchor body 1 made of PEEK (Polyether Ether Ketone). An interior of the anchor body 1 is provided with a through hollow cavity 11 in an axial direction thereof. In a direction from the tail to the head of the anchor body 1, the anchor body 1 is divided into an upper section 2, a middle section 3 and a lower section 4. Each of the upper section 2, the middle section 3 and the lower section 4 accounts for one third of a total length of the anchor body 1, and an outer diameter of the middle section 3 is smaller than that of the upper section 2.

In this embodiment, the outer diameter of the upper section 2 gradually decreases in the direction from the tail to the head of the anchor body 1, and the upper section is conical as a whole. A surface of the upper section 2 is provided with first screw threads. An end face, away from the middle section 3, of the upper section 2 is provided with a groove 21, and the groove 21 is in communication with the hollow cavity 11, enabling bone cement to flow from the groove 21 into the hollow cavity 11. The groove 21 is configured to receive an applied torque force. In this embodiment, a radial section of the groove 21 is hexagonal. The middle section 3 is provided with cylindrical screw threads, the lower section 4 is provided with hollowed-out thread teeth, and the cylindrical screw threads and the hollowed-out thread teeth form second screw threads. Inner sides of the hollowed-out thread teeth 41 are provided with multiple reinforcing ribs 2 around an axis of the anchor body 1. In an embodiment, the reinforcing ribs 42 are arranged uniformly, and the multiple uniformly distributed reinforcing ribs 42 are connected with the middle section 3 to increase the torsional strength of the anchor. In an embodiment, there are four reinforcing ribs 42.

Specifically, a joint of the upper section 2 and the middle section 3 presents a conical frustum-shaped transition, and four independent suture holes 12 are arranged between an outer surface of the conical frustum-shaped transition and an end surface of the upper section 2 in the axial direction of the anchor body 1. The four suture holes 12 are neither in communication with the groove 21 and the hollow cavity 11, nor in communication with each other.

In addition, the cylindrical screw thread and the hollowed-out thread teeth 41 are a continuous threaded structure, and a taper of the upper section 2 is smaller than that of the conical frustum-shaped transition. The bone anchor can be smoothly screwed into the bone by means of different tapers. In addition, multiple holes 31 which are in communication with the hollow cavity 11 are arranged between adjacent grooves of the cylindrical screw threads of the middle section 3, and the holes 31 facilitate the bone cement to flow out from the holes to fill a gap between the anchor and the bone, thereby enhancing the pullout strength of the anchor in cancellous bone.

Embodiment II Please referring to FIG. 1 to FIG. 3, a bone anchor is provided in this embodiment, and the bone anchor includes an anchor body 1 made of titanium alloy. An interior of the anchor body 1 is provided with a through hollow cavity 11 in an axial direction thereof. In a direction from the tail to the head of the anchor body 1, the anchor body 1 is divided into an upper section 2, a middle section 3, and a lower section 4. Each of the upper section 2, the middle section 3 and the lower section 4 accounts for one third of a total length of the anchor body 1, and an outer diameter of the middle section 3 is smaller than that of the upper section 2.

In this embodiment, the outer diameter of the upper section 2 gradually decreases in the direction from the tail to the head of the anchor body 1, and the upper section is conical as a whole. A surface of the upper section 2 is provided with first screw threads. An end face, away from the middle section 3, of the upper section 2 is provided with a groove 21, and the groove 21 is in communication with the hollow cavity 11, enabling bone cement to flow from the groove 21 into the hollow cavity 11. The groove 21 is configured to receive an applied torque force. In this embodiment, a radial section of the groove 21 is quincunx. The middle section 3 is provided with cylindrical screw threads, the lower section 4 is provided with hollowed-out thread teeth, and the cylindrical screw threads and the hollowed-out thread teeth 41 form second screw threads. Inner sides of the hollowed-out thread teeth 41 are provided with multiple reinforcing ribs 42 around an axis of the anchor body 1. In an embodiment, the reinforcing ribs 42 are arranged uniformly, and the multiple uniformly distributed reinforcing ribs 42 are connected with the middle section 3 to increase the torsional strength of the anchor. In an embodiment, there are two reinforcing ribs 42.

Specifically, a joint of the upper section 2 and the middle section 3 presents a conical frustum-shaped transition, and four independent suture holes 12 are arranged between an outer surface of the conical frustum-shaped transition and an end surface of the upper section 2 in the axial direction of the anchor body 1. The four suture holes 12 are neither in communication with the groove 21 and the hollow cavity 11, nor in communication with each other.

In addition, the cylindrical screw threads and the hollowed-out thread teeth 41 are a continuous threaded structure, and a taper of the upper section 2 is smaller than that of the conical frustum-shaped transition. The bone anchor can be smoothly screwed into the bone by means of different tapers. In addition, multiple holes 31 which are in communication with the hollow cavity 11 are arranged between adjacent grooves of the cylindrical screw threads of the middle section 3, and the holes 31 facilitate the bone cement to flow out from the holes to fill a gap between the anchor and the bone, thereby enhancing the pullout strength of the anchor in cancellous bone.

During use of the above bone anchor, end parts of one suture respectively penetrate into two adjacent suture holes at the conical frustum-shaped transition and penetrate out from the suture holes at the end surface of the upper section 2. Four suture holes 12 require two sutures. The anchor is screwed into the bone through an injection rod 80, then the tendon-ligament tissues are fixed by the suture, and finally the bone cement is injected into the anchor. The upper section 2 of the anchor is engaged with the cortical bone and the cancellous bone. The middle section 3 and the lower section 4 of the anchor are engaged with cancellous bone. The upper section 2 is designed as a conical frustum shape, which is convenient for the anchor to screw into the cortical bone with hard bone.

### Embodiment III

In the traditional fitting of the bone cement and the bone anchor, as the bone cement and the bone anchor belong to different operating systems, it is easy to take the bone anchor out of the bone when an injector is withdrawn after injection of the bone cement, which affects the fixation effect of the anchor. In view of this, this embodiment provides an implantation handle. In this embodiment, the bone anchor 10 in fit with the implantation handle is the bone anchor provided in Embodiment I or Embodiment II.

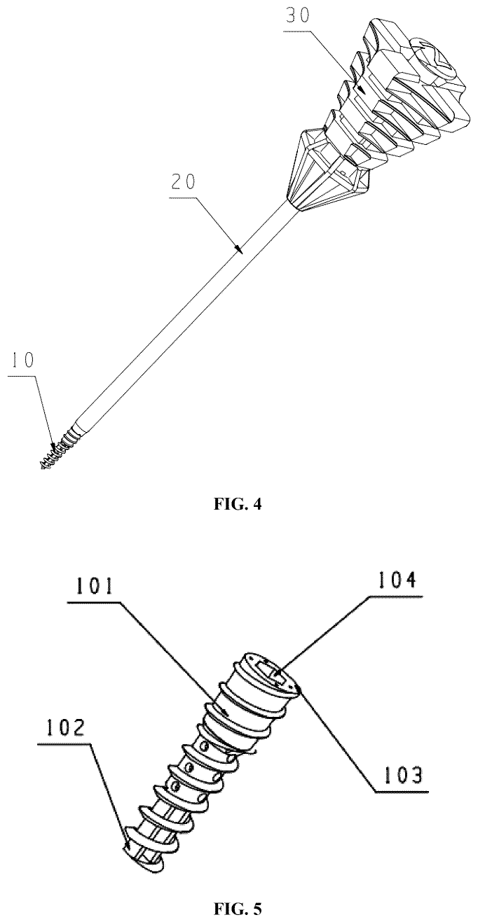

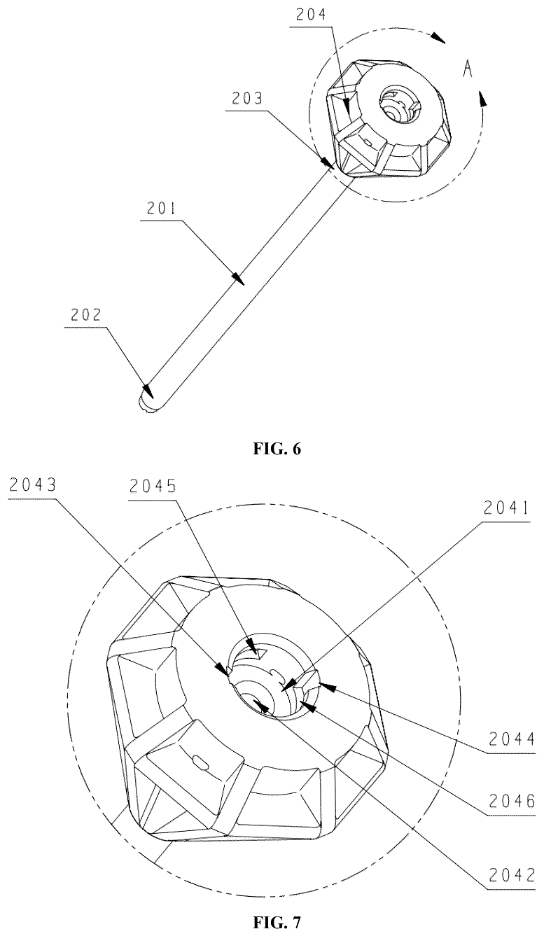

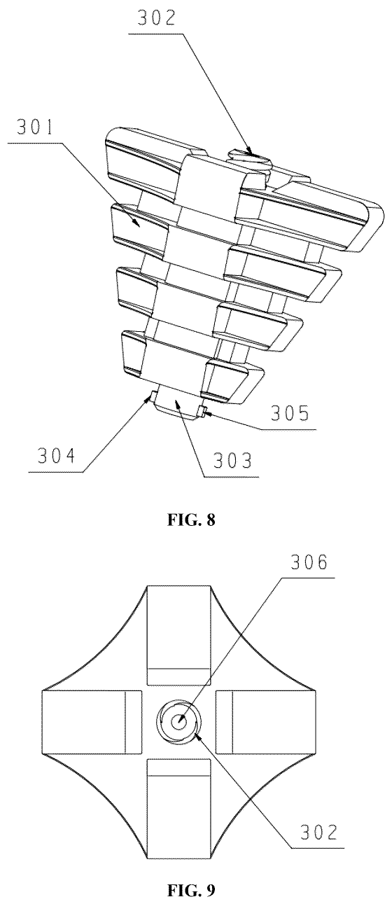

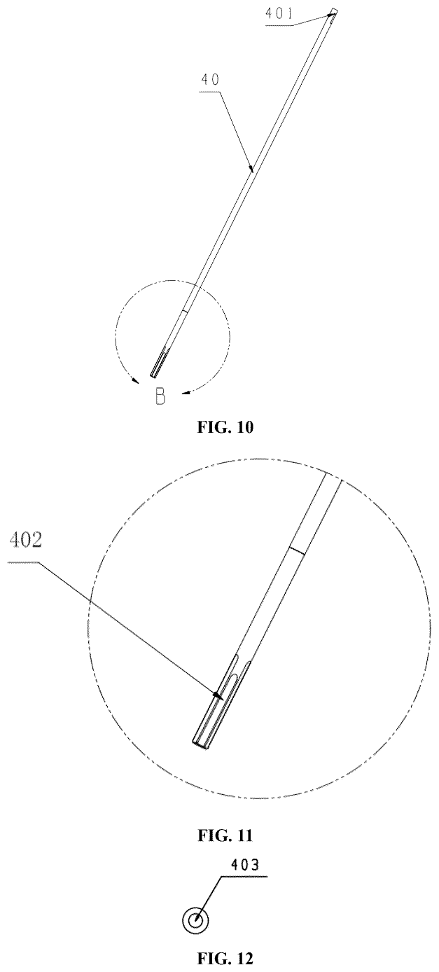

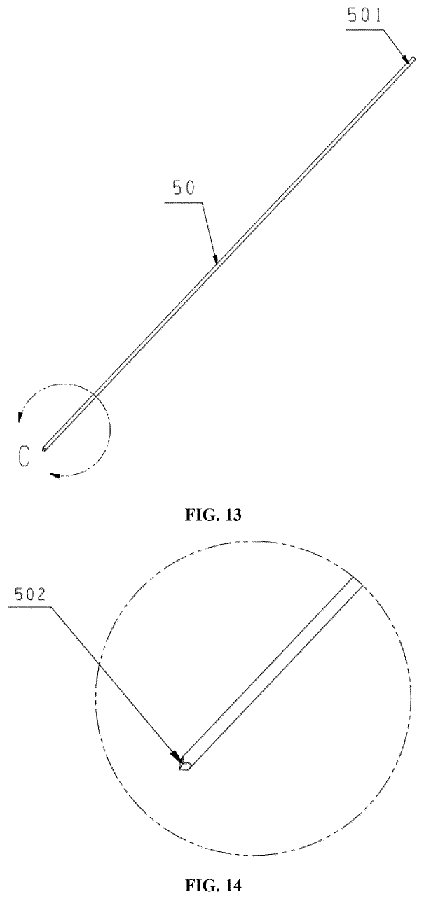

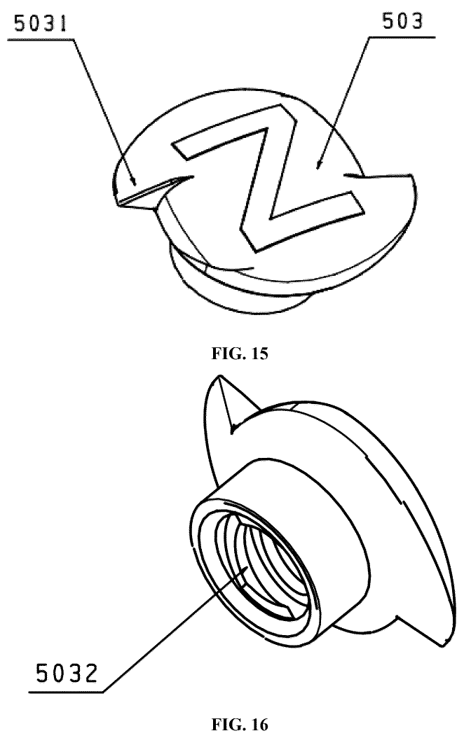

Please referring to FIG. 4 to FIG. 16, an implantation handle is provided in this embodiment, and the implantation handle includes a guide sleeve component 20, a handle component 30, a drive rod 40 and a core rod 50. The guide sleeve component 20 is provided with a guide sleeve 201 and a gripping part 204 fixedly connected with the guide sleeve. The gripping part 204 is provided with a connecting slot 2041. One end, away from the gripping part 204, of the guide sleeve 201 is provided with a non-return part 202. An outer diameter of the non-return part 202 gradually decreases in a direction away from the gripping part 204, and the non-return part 202 is conical as a whole. The conical arrangement of the non-return part 202 is convenient for observing whether the guide sleeve 201 is abutted against the bone anchor 10. A handle body 301 in the handle component 30 is fixedly connected with the drive rod 40, and the handle body 301 is provided with an engagement part 303. The engagement part 303 is detachably connected with the connecting slot 2041, and the detachable connection refers to that the engagement part 303 can slide in or out the connecting slot 2041.

Specifically, a curved sidewall of the connecting slot 2041 is provided with a first vertical guide groove 2043 and a first horizontal sliding chute 2045 which are in communication with each other, and a second vertical guide groove 2044 and a second horizontal sliding chute 2046 which are in communication with each other. The engagement part 303 of the handle body 301 is symmetrically provided with a first locking post 304 and a second locking post 305. The first locking post 304 slides into or out of the first vertical guide groove 2043 and the first horizontal sliding chute 2045. The second locking post 305 slides into or out of the second vertical guide groove 2044 and the second horizontal sliding chute 2046. It should be noted that the first vertical guide groove 2043 and the first horizontal sliding chute 2045, as well as the second vertical guide groove 2044 and the second horizontal sliding chute 2046 are two groups of symmetric structures.

In an embodiment, one end, away from the engagement part 303, of the handle body 301 is provided with an external threaded connecting part 302. One end, away from the engagement part 303, of the drive rod 40 is provided with a driving part 402. The drive rod 40 is fixedly connected or integrally formed with the handle body 301, and the driving part 402 is configured to drive the bone anchor to screw into the bone. The center of the drive rod 40 is provided with a through guide hole 403 in an axial direction thereof. The core rod 50 is provided with an operation cap 503, and the operation cap 503 is provided with an internal threaded connecting part 5032. One end, away from the operation cap 503, of the core rod 50 is provided with an inserting tip 502. The operation cap 503 is fixedly connected or integrally formed with the core rod 50, the operation cap 503 is Z-shaped as a whole, and the operation cap 503 has two operation protruding ends. The inserting tip 502 of the core rod 50 plays a role of guiding during the implantation of the bone anchor 10. In addition, when the injection amount of the bone cement is less, the bone cement in an injection rod can be completely pushed into the bone anchor 10 by using the core rod 50.

During use, the implantation handle is assembled at first, the first locking post 304 and the second locking post 305 on the engagement part 303 of the handle body 301 respectively enter the first horizontal sliding chute 2045 and the second horizontal sliding chute 2046 through the first vertical guide groove 2043 and the second vertical guide groove 2044 and slide a certain distance. Under the action of sidewalls of the two horizontal sliding chutes, the first locking post 304 and the second locking post 305 are limited. At this time, the drive rod 40 is inserted into the guide sleeve 201, the driving part 402 of the drive rod 40 is connected to a groove 104 of the anchor body 101, and the non-return part 202 of the guide sleeve 201 is abutted against the tail 103 of the anchor. Finally, the core rod 50 is inserted into the drive rod 40, the inserting tip 502 of the core rod 50 is exposed from the anchor head 102 to facilitate the implantation of the bone anchor 10, and the internal threaded connecting part 5032 of the operation cap 503 is connected to the external threaded connecting part 302 of the handle body 301. After the assembling is completed, all the above components are fixed together, and the bone anchor 1 can be screwed into the bone by operating the handle body 301.

The bone cement is injected after above operation is completed. Firstly, the operation cap 503 is separated from the handle body 301 by rotating the operation cap 503, and the core rod 50 is taken out from the drive rod 40 by using the operation cap 503. An internal threaded interface of an injector is connected with an external threaded connecting part 302 of the handle body 301, the bone cement is injected into the bone through the guide hole 403 of the drive rod 40 and the bone anchor 10 under the action of the injector. The injector is taken off after the injection is completed, and then the handle component 30 is taken off. Specifically, the handle body 301 is held immobile with one hand, and the gripping part 204 is operated with the other hand, enabling the first locking post 304 and the second locking post 305 on the engagement part 303 of the handle body 301 to move horizontally along the first horizontal sliding chute 2045 and the second horizontal sliding chute 2046, respectively. The first locking post 304 and the second locking post 305 are moved until the first locking post 304 and the second locking post 305 are aligned with the first vertical guide groove 2043 and the second vertical guide groove 2044, respectively. Then the gripping part 204 is held immobile with one hand, and the handle body 301 is moved upwards with the other hand, the first locking post 304 and the second locking post 305 of the handle body 301 move upwards along the first vertical guide groove 2043 and the second vertical guide groove 2044, respectively, thus separating the gripping part 204 from the handle body 31. The drive rod 40 is removed from the guide sleeve 201 along with the handle body 301, and in the process of removing the drive rod 40, the non-return part 202 of the guide sleeve 201 is still in contact with the groove 21 at the upper section 2 of the bone anchor 10, thus preventing the drive rod 40 from taking the bone anchor 10 out from the bone when withdrawn with the handle component 30.

Any modification, equivalent substitution, improvement, etc. made within the spirit and principle of the present disclosure should be included in the scope of protection of the present disclosure.

What is claimed is:

1. A bone anchor, comprising an anchor body (1), wherein an interior of the anchor body (1) is provided with a through hollow cavity (11) in an axial direction thereof; in a direction from a tail to a head of the anchor body (1), the anchor body (1) is divided into an upper section (2), a middle section (3) and a lower section (4); and an outer diameter of the middle section (3) is smaller than an outer diameter of the upper section (2);
the outer diameter of the upper section (2) gradually decreases in the direction from the tail to the head of the anchor body (1), and the upper section (2) is conical as a whole; a surface of the upper section (2) is provided with first screw threads; an end face, away from the middle section (3), of the upper section (2) is provided with a groove (21), and the groove (21) is in communication with the hollow cavity (11);
the middle section (3) is provided with cylindrical screw threads, the lower section (4) is provided with hollowed-out thread teeth (41), and the cylindrical screw threads and the hollowed-out thread teeth form second screw threads; and
inner sides of the hollowed-out thread teeth (41) are provided with a plurality of reinforcing ribs (42) around an axis of the anchor body (1).

2. The bone anchor according to claim 1, wherein the cylindrical screw threads and the hollowed-out thread teeth (41) form a continuous threaded structure.

3. The bone anchor according to claim 1, wherein a joint of the upper section (2) and the middle section (3) presents a conical frustum-shaped transition, and four independent suture holes (12) are arranged between an outer surface of the conical frustum-shaped transition and an end surface of the upper section (2) in the axial direction of the anchor body (1).

4. The bone anchor according to claim 1, wherein each of the upper section (2), the middle section (3) and the lower section (4) accounts for one third of a total length of the anchor body (1), and the plurality of reinforcing ribs (42) are connected with the middle section (3).

5. The bone anchor according to claim 1, wherein the plurality of reinforcing ribs (42) are uniformly arranged.

6. The bone anchor according to claim 1, wherein a plurality of holes (31) in communication with the hollow cavity (11) are arranged between adjacent grooves of the cylindrical screw threads of the middle section (3).

7. The bone anchor according to claim 1, wherein the plurality of reinforcing ribs (42) comprise two or four reinforcing ribs.

8. The bone anchor according to claim 1, wherein a radial section of the groove (21) is quincunx or hexagonal.

9. The bone anchor according to claim 3, wherein a taper of the upper section (2) is smaller than a taper of the conical frustum-shaped transition.

10. An implantation handle, comprising a guide sleeve component (20), a handle component (30), and a drive rod (40); wherein
the guide sleeve component (20) is provided with a guide sleeve (201) and a gripping part (204) fixedly connected with the guide sleeve, the gripping part (204) is provided with a connecting slot (2041); and one end, away from the gripping part (204), of the guide sleeve (201) is provided with a non-return part (202);
a handle body (301) in the handle component (30) is fixedly connected with the drive rod (40), the handle body (301) is provided with an engagement part (303), and the drive rod (40) is inserted into the guide sleeve (201); and
the engagement part (303) is detachably connected with the connecting slot (2041).

11. The implantation handle according to claim 10, wherein a curved sidewall of the connecting slot (2041) is provided with a first vertical guide groove (2043) and a first horizontal sliding chute (2045) which are in communication with each other, and a second vertical guide groove (2044) and a second horizontal sliding chute (2046) which are in communication with each other.

12. The implantation handle according to claim 11, wherein the engagement part (303) of the handle body (301) is provided with a first locking post (304) and a second locking post (305);
the first locking post (304) slides into or out of the first vertical guide groove (2043) and the first horizontal sliding chute (2045); and the second locking post (305) slides into or out of the second vertical guide groove (2044) and the second horizontal sliding chute (2046).

13. The implantation handle according to claim 10, wherein one end, away from the engagement part (303), of the drive rod (40) is provided with a driving part (402).

14. The implantation handle according to claim 13, wherein a center of the drive rod (40) is provided with a through guide hole (403) in an axial direction thereof.

15. The implantation handle according to claim 10, wherein an outer diameter of the non-return part (202) gradually decreases in a direction away from the gripping part (204), and the non-return part (202) is conical as a whole.

16. The implantation handle according to claim 10, wherein one end, away from the engagement part (303), of the handle body (301) is provided with an external threaded connecting part (302).

17. The implantation handle according to claim 16, wherein the implantation handle further comprises a core rod (50), the core rod (50) is configured to be insertable into the drive rod (40), the core rod (50) is provided with an operation cap (503), and the operation cap (503) is provided with an internal threaded connecting part (5032).

18. The implantation handle according to claim 17, wherein one end, away from the operation cap (503), of the core rod (50) is provided with an inserting tip (502).

19. An implantation system, comprising the implantation handle according to claim 10.
